(19) United States
(12) Patent Application Publication
(10) Pub. No.: US 2014/0066765 A1
(43) Pub. Date: Mar. 6, 2014

(54) METHODS AND SYSTEMS FOR INTRAVASCULAR IMAGING AND FLOW MEASUREMENT

(71) Applicants: Ying Fan, Niskayuna, NY (US); Hao Lai, Niskayuna, NY (US)

(72) Inventors: Ying Fan, Niskayuna, NY (US); Hao Lai, Niskayuna, NY (US)

(73) Assignee: GENERAL ELECTRIC COMPANY, Schenectady, NY (US)

(21) Appl. No.: 13/600,746

(22) Filed: Aug. 31, 2012

(51) Int. Cl. A61B 6/00; A61B 8/06; A61B 5/0215; A61B 5/01; A61B 5/026; A61B 8/12

(52) U.S. Cl. 600/440; 600/407; 600/479; 600/454

(57) ABSTRACT

Methods, systems and non-transitory computer readable media that store instructions executable by one or more processors for performing an interventional procedure are presented. One or more pulses are delivered to an intravascular region of interest (ROI) in a subject using at least one image sensor and at least one forward-looking flow sensor disposed at a distal end of an integrated intravascular device. Further, one or more images corresponding to the ROI are reconstructed using imaging signals received in response to the pulses delivered by the image sensor. Additionally, one or more flow characteristics corresponding to the ROI are determined based on the signals received in response to the pulses delivered by the flow sensor. The determined flow characteristics are used for computing one or more functional parameters corresponding to the ROI. An assessment of the subject may be provided based on the reconstructed images and/or the functional parameters.

### BACKGROUND

Interventional techniques are widely used for managing a plurality of life-threatening medical conditions. Particularly, certain interventional techniques entail minimally invasive image-guided procedures that provide a cost-effective alternative to invasive surgery. Intravascular Ultrasound (IVUS) imaging, for example, may be employed as a minimally invasive technique for diagnosing blocked blood vessels to provide information that aid medical practitioners in procedures such as angiography and stent placement to restore or increase blood flow to a desired region. Further, IVUS imaging systems may be used to determine existence as well as nature and extent of intravascular obstructions, stenosis or atheromatous plaque build-up at particular locations within the blood vessels.

To that end, IVUS imaging entails use of a miniaturized ultrasound probe typically including a catheter of about 1 millimeter (mm) in size that may be inserted into, or proximal, a region of interest (ROI) such as a coronary vessel. Particularly, the IVUS catheter may include an imaging sensor such as a side-looking transducer for generating high-frequency sound waves that reflect off tissue or vessel walls. The reflected sound waves may be used to generate cross-sectional images from within the vessel for visualizing structural aspects of the vessel in offline or real-time mode. The IVUS images may then be used, for example, to provide a determination of percent area stenosis for estimating severity of a stenotic lesion.

Revascularization of stenotic lesions that induce ischemia may improve a patient's functional status and outcome. Conversely, for stenotic lesions that do not induce ischemia, medical therapy alone may be as effective as revascularization without involving any of the associated risks. Estimation of structural characteristics of a stenotic lesion alone, however, may not provide sufficient information for determining which lesions cause ischemia and warrant revascularization or stenting. Accordingly, functional parameters such as fractional flow reserve (FFR) may be estimated for assessing a hemodynamic significance of the stenotic lesion, for example, for determining a likelihood of the stenotic lesion impeding oxygen delivery to the heart muscle of the patient.

FFR may be defined as a pressure behind or distal to a stenosis relative to a pressure preceding the stenosis. Alternatively, FFR may be defined as a maximal flow of a fluid down a vessel in the presence of the stenosis compared to a maximal fluid flow in the hypothetical absence of the stenosis. In one example, an FFR value less than 0.75 may be used as indication of a stenosis severity that may necessitate stenting. Typically, an FFR catheter of about 300 micron in size may be advanced across the stenosis to determine a ratio of distal coronary pressure to aortic pressure. The determined ratio may then be used to estimate functional parameters such as FFR and/or pressure corresponding to the blood flow in a vascular structure of interest.

Further, the functional parameters may be used in conjunction with the structural characteristics determined using IVUS imaging, for example, for facilitating revascularization decisions. The IVUS imaging aids in planning and/or post-procedure evaluation of the revascularization procedure by providing information regarding vessel size, lesion distribution and/or lesion type. Similarly, the FFR estimation provides a functional evaluation during the procedure planning and post-procedure evaluation for determining the efficacy of the revascularization process.

Interventional procedures, thus, may entail use of two different systems that allow for IVUS imaging and FFR estimations. Alternatively, certain systems that incorporate the IVUS and FFR catheters into a single system with integrated back-end processing of the data acquired by the IVUS and FFR catheters may be employed. Use of such systems, however, entails multiple insertions and retractions of the IVUS and FFR catheters for measuring structural and functional parameters, respectively. The multiple insertions and retractions may result in deviation from a desired measurement site in the vessel, longer procedure time and patient discomfort. Further, advancing the FFR catheter across the stenosis may impede blood flow, thus leading to erroneous measurements. Additionally, use of the different IVUS and FFR catheters may also add to equipment and operational costs.

### BRIEF DESCRIPTION

Certain aspects of the present disclosure are drawn to exemplary methods, systems and non-transitory computer readable media that store instructions executable by one or more processors for performing an interventional procedure. One or more pulses are delivered to an intravascular ROI in a subject using at least one image sensor and at least one forward-looking flow sensor disposed at a distal end of an integrated intravascular device. Further, one or more images corresponding to the ROI are reconstructed using one or more imaging signals received in response to the one or more pulses delivered by the image sensor. Additionally, one or more flow characteristics corresponding to the ROI are determined based on the one or more signals received in response to the one or more pulses delivered by the flow sensor. The determined flow characteristics, in turn, are used for computing one or more functional parameters corresponding to the ROI. An assessment of the subject may then be provided based on one or more of the reconstructed images and the one or more functional parameters.

One technical effect of the embodiments of methods, systems and non-transitory computer readable media of the present disclosure includes simplifying workflow of an interventional procedure by combining an IVUS and an FFR catheter into a single integrated intravascular device. Certain other technical effects of the present disclosure include providing more accurate lesion assessment based on accurate flow volume and velocity estimation and simultaneous measurement of structural and functional information corresponding to a target ROI using a single catheter.

### DRAWINGS

These and other features, aspects, and advantages of the present disclosure will become better understood when the following detailed description is read with reference to the accompanying drawings in which like characters represent like parts throughout the drawings, wherein:

FIG. 1 is a schematic representation of an exemplary imaging system, in accordance with aspects of the present disclosure;

FIG. 2 is a schematic representation of an exemplary embodiment of an integrated intravascular device for use in intravascular imaging and flow measurement, in accordance with aspects of the present disclosure; and

FIG. 3 is a flow diagram illustrating an exemplary method for performing an interventional procedure, in accordance with aspects of the present disclosure.

### DETAILED DESCRIPTION

The following description presents systems and methods for combining flow measurement with intravascular imaging using an integrated intravascular device. Particularly, certain embodiments illustrated herein describe methods and systems that use the integrated intravascular device for acquiring patient data that allows for simultaneous intravascular imaging and functional assessment of a vascular structure of a subject. Specifically, embodiments of the present methods and the systems use the acquired data for accurately characterizing the structural and functional parameters of the vascular structure. The accurate characterization, in turn, provides more reliable information as compared to conventional interventional systems for substantially improving interventional procedure planning, execution and post-procedure evaluation.

Although the following description is discussed with reference to IVUS imaging, certain embodiments of the present methods and systems may also be implemented in connection with other types of catheter-based interventional imaging systems, such as Optical Coherence Tomography (OCT) systems and Intra-cardiac Echocardiography (ICE) systems. Particularly, the systems and methods described herein find use, for example, in improving detection of coronary artery lesion and other anomalies in heart, thyroid, liver or other organs of the subject. The present systems and methods may also be used to more accurately diagnose and stage coronary artery disease and to help monitor therapies including, high-intensity focused ultrasound (HIFU), radiofrequency ablation (RFA) and brachytherapy using more accurate structural and functional measurements.

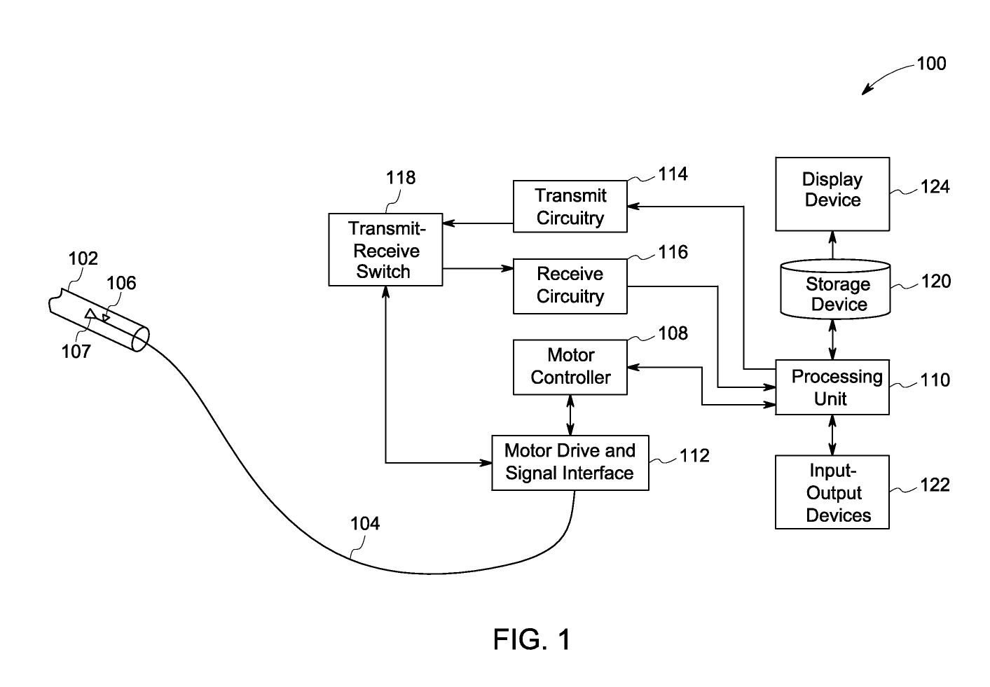

In certain embodiments, the present systems and methods may also be used for non-medical purposes, such as for nondestructive testing of fluid delivery systems, leak detection and estimation of differential pressure measurement. An exemplary environment that is suitable for practicing various implementations of the present system is described in the following sections with reference to FIG. 1.

FIG. 1 illustrates an exemplary intravascular imaging system 100, for example, for use in intravascular imaging, providing a functional evaluation and/or therapy to one or more target locations in biological tissues of interest. For discussion purposes, the system 100 is described with reference to an IVUS system. However, as previously noted, in certain embodiments, the system 100 may be implemented as other intravascular imaging systems such as an OCT and an ICE system. Additionally, it may be noted that although the present embodiment is described with reference to a blood vessel 102, certain embodiments of the system 100 may be used with other biological tissues such as lymph vessels, cerebral vessels and/or other objects suitable for ultrasound imaging and flow measurement.

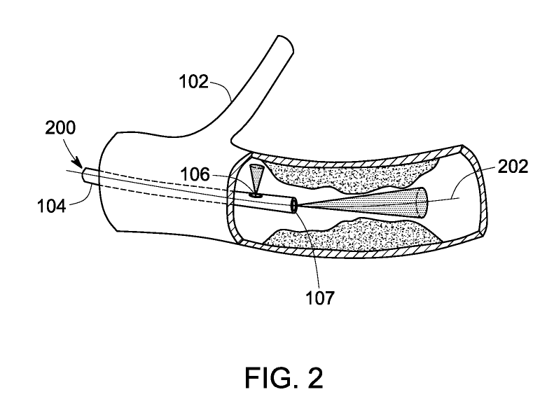

To that end, in one embodiment, the system 100 includes an integrated intravascular device such as a catheter 104 adapted for use in a confined medical or surgical environment such as a body cavity, orifice or the blood vessel 102. The catheter 104 may further include at least one image sensor 106 and at least one forward-looking flow measurement device or sensor 107 for generating cross-sectional images of the blood vessel 102 and evaluating one or more characteristics of the blood flow, respectively. The structure and functioning of the catheter 104, the image sensor 106 and the flow sensor 107 will be described in greater detail with reference to FIG. 2.

In one embodiment, the catheter 104 may be inserted into the blood vessel 102 to be imaged through one or more small incisions for reducing patient recovery time. To that end, the image sensor 106, for example, may be configured to rotate inside the blood vessel 102 under control of a motor controller 108 and/or a processing unit 110 through a motor drive and signal interface 112. Additionally, the motor controller 108 and/or the processing unit 110 may also control operation of the forward-looking flow sensor 107 to generate ultrasound pulses of a desired frequency and repetition rate for use in estimating blood flow characteristics. Particularly, the motor controller 108 and/or the processing unit 110 may provide control and timing signals for controlling various imaging parameters for either or both of the image sensor 106 and the flow sensor 107. The imaging parameters, for example, may include controlling a delivery sequence of the different ultrasound pulses, frequency of delivering the pulses, a time delay between two different pulses, beam intensity, and/or other imaging system parameters.

In certain embodiments, the system 100 further includes transmit circuitry 114 and receive circuitry 116, which may be electrically coupled to the image sensor 106 and/or the flow sensor 107 through a transmit-receive switch 118 and the motor drive and signal interface 108. In one embodiment, the transmit circuitry 114, under control of processing unit 110, generates a pulsed electrical signal to drive the image sensor 106 and/or the flow sensor 107 to emit ultrasound energy into a desired ROI (not shown). At least a portion of the emitted ultrasound energy is reflected from one or more scatterers in the ROI to produce reflected ultrasound energy that returns to the image sensor 106 and the flow sensor 107, which in turn convert the reflected ultrasound energy into electrical signals. These electrical signals pass through the motor drive and signal interface 112 and/or the transmit-receive switch 118 to the receive circuitry 116 for further processing.

Particularly, in one embodiment, the received electrical signals are provided to the processing unit 110 that processes the received signals according to a plurality of selectable ultrasound modalities in real-time and/or off-line mode. To that end, the processing unit 110 includes devices such as one or more application-specific processors, digital signal processors, microcomputers, microcontrollers, Application Specific Integrated Circuits (ASICs) and/or Field Programmable Gate Arrays (FPGAs). The processing unit 110 may also include devices in communication with other components of the system 100 such as a picture archiving and communications system (PACS), a radiology department information system, hospital information system and/or to an internal or external network (not shown).

In certain embodiments, the processing unit 110 stores the received signals and/or processed information along with the delivery sequence, repetition frequency, time delay, intensity, imaging system parameters and/or other operational data in a storage device 120 for further processing. To that end, the storage device 120 may include devices such as a random access memory, a read-only memory, a disc drive, a solid-state memory device, and/or a flash memory. Additionally, the processing unit 110 may communicate the processed information to the motor controller 108 operatively coupled to a motor (not shown) by the motor drive and signal interface 112.

In one embodiment, the motor controller 108 uses the information received from the processing unit 110 to configure the image sensor 106 to rotate about the longitudinal axis of the catheter 104, for example, via a rotatable driveshaft. Particularly, the motor controller 108 controls a rate of rotation of the image sensor 106 based on specific imaging requirements. To that end, in certain embodiments, the motor controller 118 configures the motor drive and signal interface 112 to rotate the image sensor 106 while allowing electrical signals to pass between the rotating image sensor 106 and the stationary components of the system 100. Although FIG. 1 illustrates the motor controller 108 as an independent entity, in certain embodiments, the motor controller 108 may be implemented as part of the processing unit 110.

Further, in certain embodiments, the processing unit 110 is coupled to one or more user input-output devices 122, such as a keyboard, touchscreen, microphone, mouse, buttons, and/or switches for receiving commands and inputs from an operator. In one example, the processing unit 110 allows the operator to select one or more imaging regions of interest and/or imaging parameters, for example, using a graphical user interface on a local or remote display device 124 communicatively coupled to the processing unit 110 and/or the input output devices 122. The imaging parameters, for example, may include a rate of rotation of the imaging sensor 106, a velocity or length of the pullback of the catheter 104, a selection of functional parameters for display and one or more desired properties of the images.

The processing unit 110, in one embodiment, conveys operator inputs to one or more of the transmit circuitry 114 and the motor controller 108. Accordingly, the image sensor 106, rotating under the control of the motor controller 108, emits ultrasound pulses towards one or more desired portions of the region surrounding the image sensor 106 to allow generation of a plurality of imaging lines. These imaging lines may be used collectively to reconstruct a radial cross-sectional image of the desired ROI, such as the walls of the blood vessel 102 and the tissue surrounding the blood vessel 102.

In one example, the processing unit 110 displays corresponding patient data including ultrasound images for review, diagnosis, analysis, and treatment. In another example, the processing unit 110 stores the ultrasound images for later review and analysis, or communicates the images to another location for further review. Further, in one embodiment, a medical practitioner employs the generated images, for example, for detecting a pathological condition such as presence of plaque or other blockages and/or to aid in deploying a vascular stent.

However, as previously noted, structural information derived from the generated images may not be sufficient for identifying ischemia-causing lesions and/or for making certain other procedure-related decisions. Accordingly, the processing unit 110 may be configured to provide control and timing signals to the forward-looking flow sensor 107 for generating a repetitive sequence of pulses that may be transmitted along the same scan line for generating M-mode data corresponding to a flow of a fluid such as blood through the imaged blood vessel 102. In certain embodiments, the flow sensor 107 is configured to acquire the functional data simultaneously with the imaging data. In certain other embodiments, the imaging data and the functional data may be acquired at different instants of time based on user input and/or control signals received from the processing unit 110.

The processing unit 110 may then estimate functional parameters for the target ROI based on the received signals. By way of example, for intensity M-mode data, each received signal along a particular scan line may be used for determining intensities. For color M-mode data, the processing unit 110 may evaluate a group of transmitted and received signals along the scan line for estimating flow characteristics such as velocity, volume and/or pressure. The processing unit 110 may then combine the structural information acquired using the image sensor 106 with the functional information acquired using the flow sensor 107 to provide a more informed assessment of the target ROI.

Although FIG. 1 illustrates certain exemplary components, in certain embodiments, the system 100 may include fewer or additional components for use in intravascular imaging, acquired data processing and/or for allowing automation of the interventional procedure. In one example, the system 100 may include additional devices such as one or more analog-to-digital-converters (ADC), filters, amplifiers and/or switching subsystems. Alternatively, one or more of the components such as the motor controller 108, the processing unit 110, the transmit circuitry 114 and/or the receive circuitry 116 may be combined into a single or fewer devices, thus optimizing floor space in the interventional room.

Embodiments of the present system 100, thus, provide enhanced functionality and operational efficiency, while allowing the interventional practitioner and/or the automated system 100 to perform the interventional procedure with greater reliability. Particularly, use of the integrated catheter 104 including both the image sensor 106 and the flow sensor 107 simplifies the workflow of the interventional procedure, which in turn, aids in reducing the procedure time and improving data accuracy and patient comfort. The structure and a functioning of the integrated catheter 104 for use in efficient interventional procedures will be described in greater detail with reference to FIG. 2.

FIG. 2 illustrates an exemplary embodiment of an integrated intravascular device 200 for use in simultaneous intravascular imaging and flow measurement. To that end, in certain embodiments, the device 200 may include a catheter, such as the catheter 104 of FIG. 1. As previously noted with reference to FIG. 1, the catheter 104 may further include at least one image sensor 106 and the at least one flow sensor 107. In one embodiment, the catheter 104, for example, may be a mechanical or a phased-array catheter including a manual and/or a mechanized pullback mechanism (not shown).

Further, in certain embodiments, the catheter 104 may be operatively coupled to a guide wire 202 for aiding insertion of the catheter 104 into an access site proximal or distal from the intravascular ROI (target ROI). To that end, the guide wire 202, for example, may be a thin wire with a flexible tip that may be guided through the blood vessel 102 proximal to or across the blockage or stenosis to allow for imaging and/or flow measurements. In certain embodiments, the interventional practitioner controls the movement and direction of the guide wire 202 by gently manipulating the end of the guide wire 202 that sits outside the patient. Particularly, the interventional practitioner manipulates the guide wire so as to pass the catheter 104 over the guide wire 202 and position the distal end of the catheter 104 close to the lesion or blockage.

The insertion and movement of the catheter 104 within the blood vessel 102, however, is a challenging procedure. Accordingly, in certain embodiments, the integrated device 200 may be communicatively coupled to an interventional imaging system and/or a tracking system (not shown in FIG. 2) for providing guidance for navigating the catheter 104 and/or the guide wire 202 through the blood vessel 102 without injuring the surrounding tissues. Additionally, when traversing a tortuous vessel, as previously noted, the distal end of the catheter 104 may be positioned proximal the target ROI such as close to a site of the stenosis. Once positioned, the image sensor 106 and/or the flow sensor 107 in the catheter 104 may be configured to transmit and receive ultrasound signals along one or more scan lines for imaging surrounding tissues and determining blood flow characteristics, respectively.

To that end, in one embodiment, the image sensor 106 may be a side-looking ultrasound transducer positioned at the distal end of the catheter 104 and configured to generate a two-dimensional (2D) and/or a three-dimensional (3D) cross-sectional image of the stenotic portion of the blood vessel 102. Particularly, the image sensor 106 may include a single-element miniaturized transducer configured to rotate mechanically about the catheter 104, offering side-looking capabilities. The size of the transducer, in one example, may be in the range of 0.25 to 1.0 mm. Further, the transducer may be flat or curved, disc-, block-, spherical- or ring-shaped based on specific imaging requirements. In certain embodiments, the shape and size of the transducer may be selected such that the image sensor 106 is suitable for being inserted or placed inside a patient's body without significant tissue disruption.

Although FIG. 2 depicts the image sensor 106 including a single-element transducer, in an alternative embodiment, the image sensor 106 may include a multi-element array of transducers such that the resultant imaging can provide a 2D and/or a 3D radial cross-sectional image of the vessel wall. In an alternative embodiment, instead of an ultrasound transducer, the image sensor 106 may include an optical device suitable for optical imaging. Further, in certain embodiments, the image sensor 106 may be configured to produce a forward-looking image and/or a side-looking image of the imaged vessel. To that end, the integrated device 200 may include electric and/or mechanical steering means to allow multiple degrees of freedom for a distal tip of the catheter 104. Particularly, using the steering means, the image sensor 106 may transmit and receive ultrasound signals along one or more scan lines for acquiring imaging data from the target ROI.

Similarly, the flow sensor 107 may also use the steering means to acquire, for example, color M-mode data corresponding to the region proximal the stenosis. To that end, in one embodiment, the flow sensor 107 may be a forward-looking ultrasound Doppler sensor configured to acquire the M-mode data to determine one or more characteristics of the blood flow, such as blood volume and velocity, while traversing the stenosis. In another embodiment, however, the flow sensor 107 may include certain other flow measurement devices such as pressure sensors, temperature sensors, flow nozzles, venture tubes and/or orifice plates. An associated processing system, such as the processing unit 110 of FIG. 1, may then use the determined velocity information to compute a pressure gradient across the stenosis in the blood vessel 102.

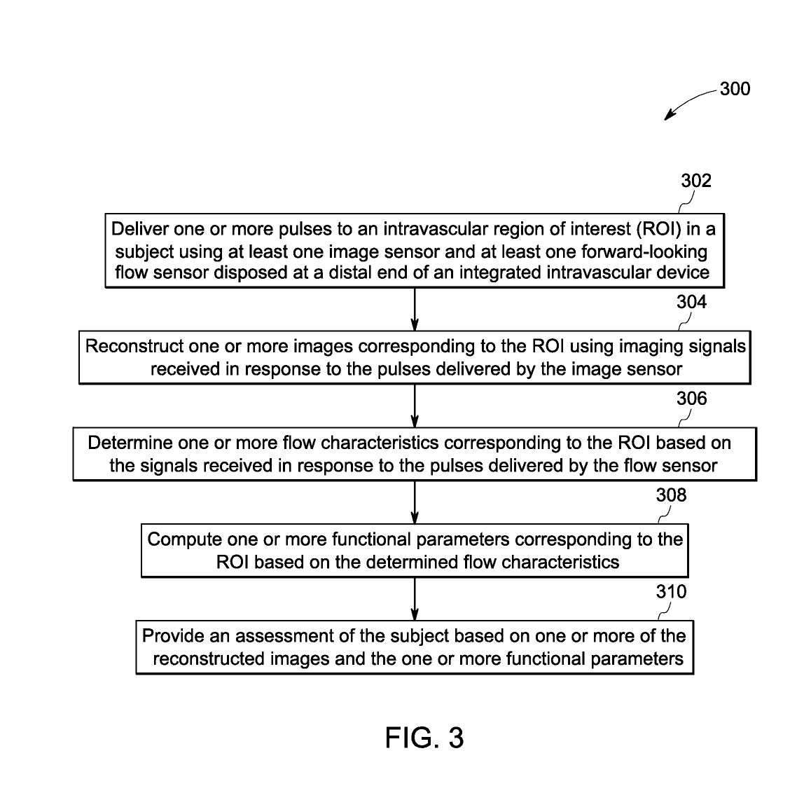

In accordance with certain aspects of the present disclosure, the pressure gradient across the stenosis may be ascertained with greater accuracy without the use of a separate FFR catheter that may typically impede the flow in conventional implementations, while also adding to procedure time. Particularly, the integrated intravascular device 200 may allow simultaneous imaging and flow measurement to provide the interventional practitioner with reliable real-time structural and/or functional information for planning, executing and evaluating the interventional procedure with greater accuracy. Certain exemplary methods for such simultaneous imaging and flow measurement using the integrated intravascular device are discussed in greater detail with reference to FIG. 3.

FIG. 3 illustrates a flow chart 300 depicting an exemplary method for performing an interventional procedure. The exemplary method may be described in a general context of computer executable instructions stored and/or executed on a computing system or a processor. Generally, computer executable instructions may include routines, programs, objects, components, data structures, procedures, modules, functions, and the like that perform particular functions or implement particular abstract data types. The exemplary method may also be practiced in a distributed computing environment where optimization functions are performed by remote processing devices that are linked through a wired and/or wireless communication network. In the distributed computing environment, the computer executable instructions may be located in both local and remote computer storage media, including memory storage devices.

Further, in FIG. 3, the exemplary method is illustrated as a collection of blocks in a logical flow chart, which represents operations that may be implemented in hardware, software, or combinations thereof. The various operations are depicted in the blocks to illustrate the functions that are performed, for example, during signal transmission and reception, data acquisition, processing and image reconstruction phases of the exemplary method. In the context of software, the blocks represent computer instructions that, when executed by one or more processing subsystems, perform the recited operations.

The order in which the exemplary method is described is not intended to be construed as a limitation, and any number of the described blocks may be combined in any order to implement the exemplary method disclosed herein, or an equivalent alternative method. Additionally, certain blocks may be deleted from the exemplary method or augmented by additional blocks with added functionality without departing from the spirit and scope of the subject matter described herein. For discussion purposes, the exemplary method will be described with reference to the elements of FIGS. 1-2.

Generally, investigation of a stenosis entails both structural and functional assessment of an affected region. Typically, the structural assessment may be performed using angiography, IVUS imaging and/or optical imaging. The measured structural characteristics may be used, for example, to compute percent occlusion of the blood vessel 102 of FIG. 1. The computed occlusion values may then be employed as an indication of stenosis severity. However, as previously noted, a more reliable determination of the nature and extent of the stenosis entails use of functional information including pressure and flow characteristics prevalent at the site of the stenosis. Certain conventional approaches, thus, have been known to employ FFR estimates to ascertain if the detected stenosis warrants stenting.

Conventional interventional systems, however, employ separate IVUS and FFR catheters for measuring structural and functional parameters, respectively. Use of such conventional interventional systems, however, entails multiple insertions and retractions of separate IVUS and FFR catheters, which may result in deviation from a desired measurement site in the vessel, thus causing measurement errors. Additionally, the multiple measurements using the different catheters may also lead to longer procedure time, greater patient discomfort and equipment costs.

Accordingly, embodiments of the present method describe a technique for performing an interventional procedure using a single integrated intravascular device that allows for simultaneous imaging and functional measurement. For discussion purposes, an exemplary embodiment of the present method will be described with reference to an interventional procedure for ascertaining a need for revascularization at a target ROI such as a site of stenosis in a blood vessel by determining corresponding structural and functional parameters.

To that end, at step 302, one or more pulses may be delivered to an intravascular ROI (target ROI) using an integrated intravascular device, such as the catheter 104 of FIG. 1 and/or the device 200 of FIG. 2. The integrated device may include at least one image sensor, such as the image sensor 106 of FIG. 1 and at least one forward-looking flow sensor such as the flow sensor 107 of FIG. 1 disposed at the distal end of the catheter 104. In certain embodiments, the image sensor may also include one or more functional measurement devices such as pressure sensors, flow sensors and/or temperature sensors for determining specific parameters corresponding to the target ROI.

In one embodiment, a plurality of ultrasound pulses having desired characteristics may be employed for imaging the target ROI. However, in an alternative embodiment, OCT may be employed for imaging the target ROI using a plurality of optical pulses. Accordingly, the image sensor may include a side-looking ultrasound sensor and/or an optical sensor suitable for imaging the target ROI based on a specific imaging modality being used. For purposes of discussion, the present embodiment is described with reference to use of ultrasound pulses for imaging the target ROI.

In one embodiment, the image sensor 106 and the flow sensor 107 may be configured to deliver the ultrasound pulses simultaneously. However, in an alternative embodiment, the image sensor 106 and the flow sensor 107 may be configured to deliver the ultrasound pulses to the target ROI, such as a region proximal a lesion in a blood vessel, at different instants of time. As previously noted with reference to FIG. 1, the timing and frequency of delivery of the ultrasound pulses by the image sensor and/or the flow sensor may be controlled by a processing system, such as the processing system 110 based on a designated protocol and/or user specified inputs.

Further, at step 304, one or more images corresponding to the target ROI may be reconstructed using one or more imaging signals received in response to the one or more pulses delivered by the image sensor. To that end, in one embodiment, the image sensor may provide side-looking and/or forward-looking imaging capability. Specifically, the image sensor may be configured to generate high-frequency sound waves that reflect off tissue or vessel walls. The reflected sound waves may be used to create cross-sectional images from within the vessel for visualizing structural aspects of the imaged vessel in offline or real-time mode. The IVUS images may then be used, for example, to provide a determination of percent area stenosis of the vessel.

Additionally, at step 306, one or more flow characteristics corresponding to the target ROI may be determined based on the one or more signals received in response to the one or more pulses delivered by the flow sensor. To that end, in one embodiment, the flow sensor may be a forward-looking ultrasound Doppler sensor. However, in an alternative embodiment, the flow sensor may be a pressure sensor configured for directly measuring a pressure gradient across the stenosis. In certain embodiments, the processing system configures the flow sensor to transmit one or more ultrasound pulses having a desired wavelength and/or frequency at designated points of time. The echo signals received in response to these ultrasound pulses may then be processed by the processing unit to determine flow characteristics such as a velocity profile of the flow of blood through the blood vessel of interest in a time and/or a space domain.

To that end, in one embodiment, the processing unit may be configured to perform a first-stage digital filtering of the received signals for removing noise and/or slow moving objects from the acquired data, for example, using a band-pass or a wall filter. Further, the processing unit may calculate a power spectrum in a direction of transmission of the ultrasound pulses and/or obtain a phase shift difference in the direction of transmission. Additionally, the processing unit may perform a second-stage digital filtering for removing noise from the processed data. The processing unit may then determine velocity of the blood flow at a particular location in the target ROI using the calculated frequency and/or the phase shift information.

In an alternative embodiment, however, the velocity profile may be determined using the reconstructed IVUS images. To that end, the processing unit may process the ultrasound signals received in response to the pulses delivered by the image sensor. Specifically, the processing unit may be configured to perform a first-stage digital filtering of the received signals for removing noise and/or slow moving objects from the acquired data, for example, using a band-pass filter. Further, the processing unit may calculate a correlation length between transmissions of successive ultrasound pulse sequences. The velocity may then be calculated by dividing the width of an ultrasound beam by an average of correlation length and the interval between the successive ultrasound transmissions. Although, the present embodiment describes calculation of velocity information, other flow characteristics such as flow volume may also be determined using the acquired ultrasound data.

Further, at step 308, one or more functional parameters corresponding to the target ROI may be computed based on the determined flow characteristics. The functional parameters, for example, may include a pressure gradient, FFR, blood flow volume and/or coronary flow reserve (CFR). Although the flow characteristics may also be used to compute other functional parameters, the present embodiment is described with reference to the computation of a pressure gradient across a site of stenosis in the blood vessel.

Accordingly, in certain embodiments, the pressure gradient may be calculated using computational fluid dynamics modeling and/or using specific expressions that provide a correlation between the determined velocity values at the target ROI and the corresponding pressure gradient. Particularly, in one example, the pressure gradient may be defined using equation (1).

$$Δ p = ρ ( \frac{\partial u}{\partial t} + u \frac{\partial u}{\partial x} ) \qquad ( 1 )$$

where Δp corresponds to the pressure gradient, ρ corresponds to fluid density, u corresponds to velocity, t corresponds to time and x corresponds to distance along the vessel of interest.

As depicted in equation 1, the pressure gradient computation entails use of a derivative of velocity, for example, in a temporal and/or a spatial domain. Additionally, the measured velocity may be multiplied by a derivative of the measured velocity as a function of distance along a scan line. The resulting product, in turn, may be summed with the temporal derivative of the velocity. This summed value may then be multiplied with the density of the fluid flowing through the vessel to estimate the pressure gradient. Specifically, equation 1 may be used to compute the pressure gradient for each spatial location from the corresponding velocity values.

It may be noted that equation 1 provides an illustrative example for defining the pressure gradient. However, in certain embodiments, other fluid dynamics model may be used to calculate the pressure gradients. The pressure gradient, for example, may be computed using Bernoulli's equation or a presently known approach using empirically derived Young's mathematical model.

Alternatively, in certain other embodiments, the pressure gradients may be computed using one or multiple pressure sensors incorporated within the catheter 104, which may entail pressure measurements at either site of the stenosis. However, as the diameter of the IVUS catheter 104 is typically about three times larger than that of an FFR catheter, pressure measurements using the IVUS catheter 104 at either side of the stenosis may severely occlude the blood flow through the stenosis. Accordingly, in one embodiment, the reconstructed images may be used to model the blood flow and account for the occluded blood flow during the pressure gradient measurements.

Subsequently, at step 310, an assessment of the subject may be provided based on one or more of the reconstructed images and the one or more functional parameters. Particularly, the pressure gradient, along with the measured structural characteristics may be used as a reliable indication of the stenosis severity, and thus, aid in determining a need for revascularization of the blood vessel. Similarly, other functional parameters may be used to assess a health condition of the subject, such as a structural or functional anomaly in the tissues indicative of a pathological condition.

Embodiments of the present systems and methods, thus, describe use of an integrated IVUS catheter for use in simultaneous imaging and functional assessment of a subject. Particularly, use of a single catheter improves a probability of measuring the imaging and functional data at the same spot in the vessel of interest, thus allowing for a more accurate correlation between the structural and functional information. Further, combining the real-time structural and functional information in a single catheter allows for a more accurate flow and volume estimation. Additionally, use of the single integrated catheter precludes multiple insertions and retractions, thus simplifying interventional workflow, reducing the procedure time and/or enhancing patient comfort.

Moreover, integrating a forward-looking Doppler sensor into the IVUS catheter allows for flow volume and velocity measurements without having to advance the catheter across the stenosis and impeding the flow of blood through the blood vessel as experienced when using an FFR catheter. The flow measurements, in turn, may be used to estimate the pressure gradients, thus obviating need to measure pressure values on either side of a stenosis to estimate a pressure gradient across the stenosis. Additionally, the forward-looking ultrasound flow sensor may also provide information of flow channel ahead of the flow sensor to provide guidance for catheterization. Furthermore, use of the integrated catheter obviates a need for separate IVUS and FFR catheters, each of which typically cost hundreds of dollars, thus optimizing equipment and/or operational costs.

It may be noted that although specific features of various embodiments of the present systems and methods may be shown in and/or described with respect to only certain drawings and not in others, this is for convenience only. It is to be understood that the described features, structures, and/or characteristics may be combined and/or used interchangeably in any suitable manner in the various embodiments, for example, to construct additional assemblies and techniques. Furthermore, the foregoing examples, demonstrations, and process steps, for example, those that may be performed by the motor controller 108 and the processing unit 110 may be implemented by a single device or a plurality of devices using suitable code on a processor-based system.

It should also be noted that different implementations of the present disclosure may perform some or all of the steps described herein in different orders or substantially concurrently, that is, in parallel. In addition, the functions may be implemented in a variety of programming languages, including but not limited to Python, C++ or Java. Such code may be stored or adapted for storage on one or more tangible, machine-readable media, such as on data repository chips, local or remote hard disks, optical disks (that is, CDs or DVDs), solid-state drives or other media, which may be accessed by a processor-based system to execute the stored code.

While only certain features of the present disclosure have been illustrated and described herein, many modifications and changes will occur to those skilled in the art. It is, therefore, to be understood that the appended claims are intended to cover all such modifications and changes as fall within the true spirit of the present disclosure.

1. A method for performing an interventional procedure, comprising:
delivering one or more pulses to an intravascular region of interest in a subject using at least one image sensor and at least one forward-looking flow sensor disposed at a distal end of an integrated intravascular device;
reconstructing one or more images corresponding to the region of interest using one or more imaging signals received in response to the one or more pulses delivered by the image sensor;
determining one or more flow characteristics corresponding to the region of interest based on the one or more signals received in response to the one or more pulses delivered by the flow sensor;
computing one or more functional parameters corresponding to the region of interest based on the determined flow characteristics; and
providing an assessment of the subject based on one or more of the reconstructed images and the one or more functional parameters.

2. The method of claim 1, wherein delivering the one or more pulses to the intravascular region of interest in the subject using the at least one image sensor comprises delivering one or more ultrasound pulses.

3. The method of claim 1, wherein delivering the one or more pulses to the intravascular region of interest in the subject using the at least one image sensor comprises delivering one or more optical pulses.

4. The method of claim 1, wherein determining one or more flow characteristics comprises calculating a velocity profile of a fluid flowing through the intravascular region of interest in one or more of a time domain and a space domain.

5. The method of claim 4, wherein calculating the velocity profile comprises:
digitally filtering the signals received in response to the one or more pulses delivered by the flow sensor;
computing one or more of a power spectrum and a phase shift difference in a direction of transmission of the pulses; and
determining velocity of the fluid flowing through the intravascular region of interest based on the computed power spectrum, the phase shift information, or a combination thereof.

6. The method of claim 4, wherein calculating the velocity profile comprises:
digitally filtering the imaging signals received in response to the one or more pulses delivered by the image sensor;
computing a correlation length between transmission of each of the pulses; and
determining velocity of the fluid flowing through the intravascular region of interest using the computed correlation length, modeling the flow of the fluid through the intravascular region of interest based on the received imaging signals, or a combination thereof.

7. The method of claim 6, wherein determining the velocity comprises:
computing an average of the correlation length and an interval between successive transmissions of the one or more pulses; and
dividing width of an ultrasound beam in the one or more pulses by the computed average.

8. The method of claim 1, wherein computing one or more functional parameters comprises determining a fractional flow reserve value, a blood flow volume, a coronary flow reserve value, or combinations thereof, corresponding to the intravascular region of interest.

9. The method of claim 1, wherein computing one or more functional parameters comprises determining a pressure gradient corresponding to the region of interest using computational fluid dynamics modeling.

10. The method of claim 9, wherein determining the pressure gradient comprises:
computing a derivative of velocity of a fluid flowing through the region of interest measured at a particular spatial location in one or more of a temporal domain and a special domain;
calculating a product of the measured velocity and the derivative of the measured velocity as a function of distance along a scan line;
determining a sum of the product and a temporal derivative of the velocity;
computing a product of density of a fluid flowing through the intravascular region of interest and the determined sum; and
determining the pressure gradient at the spatial location using the computed product.

11. The method of claim 9, wherein the pressure gradient is determined using
$$Δ p = ρ ( \frac{\partial u}{\partial t} + u \frac{\partial u}{\partial x} ) ,$$
wherein Δp corresponds to the pressure gradient, ρ corresponds to density of a fluid flowing through the intravascular region of interest, u corresponds to velocity of the fluid, t corresponds to time and x corresponds to a distance along the intravascular region of interest.

12. The method of claim 1, wherein delivering the one or more pulses comprises delivering the one or more pulses using the flow sensor simultaneously with the one or more pulses delivered using the image sensor.

13. The method of claim 1, wherein providing an assessment of the subject comprises displaying one or more of the reconstructed images, the functional parameters, the flow characteristics and the assessment of the subject on a display device.

14. An intravascular imaging system, comprising:
an integrated intravascular catheter, comprising:
at least one image sensor adapted for use in an intravascular region of interest of a subject and configured to produce signals for use in generating one or more images of the region of interest;
at least one flow sensor configured to produce signals for use in determining one or more flow characteristics corresponding to the region of interest;
wherein one or more of the image sensor and the flow sensor are positioned at the distal end of the integrated catheter;
at least one processing unit operatively coupled to the integrated catheter and configured to:
compute one or more functional parameters corresponding to the region of interest based on the determined flow characteristics; and
provide an assessment of the subject based on one or more of the reconstructed images and the one or more functional parameters.

15. The system of claim 14, wherein the image sensor comprises a side-looking ultrasound sensor.

16. The system of claim 14, wherein the image sensor comprises an optical sensor.

17. The system of claim 14, wherein the integrated intravascular catheter comprises one or more of a pressure sensor, a flow sensor and a temperature sensor integrated into the image sensor.

18. The system of claim 14, wherein the flow sensor comprises a forward-looking Doppler flow sensor.

19. The system of claim 14, further comprising a display device configured to display one or more of the reconstructed images, the functional parameters, the flow characteristics and the assessment of the subject.

20. A non-transitory computer readable medium that stores instructions executable by one or more processors to perform a method for performing an interventional procedure, comprising:
delivering one or more pulses to a region of interest in a subject using at least one image sensor and at least one forward-looking flow sensor disposed at a distal end of an integrated intravascular device;
reconstructing one or more images corresponding to the region of interest using one or more signals received in response to the one or more pulses delivered by the image sensor;
determining one or more flow characteristics corresponding to the region of interest based on the one or more signals received in response to the one or more pulses delivered by the flow sensor;
computing one or more functional parameters corresponding to the region of interest based on the determined flow characteristics; and
providing an assessment of the subject based on one or more of the reconstructed images and the one or more functional parameters.
